SEQUENCE LISTING (4 sequences)

SEQ ID NO: 1 - DNA, 18 bp, Artificial Sequence
  Description of Artificial Sequence  Synthetic primer
aatggaggtgcagtcact

SEQ ID NO: 2 - DNA, 18 bp, Artificial Sequence
  Description of Artificial Sequence  Synthetic primer
aagagaggtgtctactgc

SEQ ID NO: 3 - DNA, 18 bp, Artificial Sequence
  Description of Artificial Sequence  Synthetic primer
aaagggggtgcaccaatt

SEQ ID NO: 4 - DNA, 18 bp, Artificial Sequence
  Description of Artificial Sequence  Synthetic primer
aagggtggtaagattgtt

(19) United States
(12) Patent Application Publication
(10) Pub. No.: US 2007/0286872 A1
(43) Pub. Date: Dec. 13, 2007

(54) Live Attenuated Nidovirus Vaccines

(75) Inventor: Mark Denison, Nashville, TN (US)

Correspondence Address: FULBRIGHT & JAWORSKI L.L.P., 600 CONGRESS AVE., SUITE 2400, AUSTIN, TX, 78701, US

(73) Assignee: Vanderbilt University, Nashville, TN (US)

(21) Appl. No.: 10/553,883

(22) PCT Filed: Apr. 22, 2004
(86) PCT No.: PCT/US04/12441

§ 371 Date: Sep. 7, 2006

Related U.S. Application Data
(60) Provisional application No. 60/464,456, filed on Apr. 22, 2003.

(51) Int. Cl. A61K 39/12; A61K 35/76; A61P 31/00

(52) U.S. Cl. 424/204.1; 424/93.6

(57) ABSTRACT

The present invention is directed live, attenuated Nidovirus vaccines, and in a particular embodiment, to coronavirus vaccines. The vaccine comprises a viral genome encoding a replicase polyprotein having at least one proteinase cleavage site that exhibits reduced or no cleavage. Such viruses show reduced.

The present application claims benefit of priority to U.S. Provisional Ser. No. 60/464,456, filed Apr. 22, 2003, the entire content of which is hereby incorporated by reference.

The government owns rights in the present invention pursuant to grant number 5RO1 A126603-15 and RO1 AI50083-O1S1 of the National Institutes of Health and National Institute Allergy Infectious Disease.

### BACKGROUND OF THE INVENTION

1. Field of the Invention

The present invention relates generally to the fields of microbiology, immunology and virology. More particularly, it concerns live, attenuated Nidovirus vaccines and methods for preventing or limiting Nidovirus infections.

2. Description of Related Art

Coronaviruses have been long known to cause important diseases in a wide variety of animal species, including humans, cattle, swine, chickens, dogs, cats and mice. Coronavirus diseases in non-human species may be quite severe, and devastating in domestic livestock such as pigs, cattle and chickens. The characterized human coronaviruses —HCoV-229E and HCoV OC43—are significant causes of upper respiratory infections, responsible for 10-35% of human colds. Studies of human coronaviruses have been limited by their lack of growth in culture from primary isolates, and by the lack, until recently, of reverse genetic approaches for their study. Thus, while the human coronaviruses are arguably two of the most economically important viruses in humans, ongoing research has been pursued only by a handful of investigators.

The emergence of a new human coronavirus associated with “severe acute respiratory syndrome” (SARS) surprised many scientists and public health officials, but has highlighted characteristics of coronaviruses well known to investigators. The coronaviruses have high rates of mutagenesis and homologous RNA recombination. In fact, template switching and recombination are essential to the normal life cycle of the viruses. In addition, the species barrier for coronaviruses has been predicted to be tenuous. Studies of coronaviruses in culture have demonstrated the ability of coronaviruses to adapt for replication in cells of different species. In addition, some studies have demonstrated that the murine coronaviruses may cause disease in primates following direct inoculation into brain. Finally, coronaviruses have been proposed, based on evolutionary studies, to have acquired genes from other viruses or cells, probably by recombination events. The emergence of a new coronavirus pathogenic for humans, by either adaptation of an animal virus, or by recombination of two coronaviruses during a confection, is consistent with these features of coronavirus evolution, replication and maintenance in populations.

Vaccine approaches for important domestic animal coronaviruses diseases, specifically the chicken avian infectious bronchitis virus (IBV), porcine transmissible gastroenteritis virus (TGEV), canine coronavirus (CCV), bovine coronavirus (BCV) and feline infectious peritonitis virus (FIPV), have been developed or attempted over the past 20 years. The approaches to vaccine development have been based on non-targeted natural attenuation, virus expression vectors, virus inactivation, recombinant viral structural proteins, and novel approaches to deliver or adjuvant vaccines. Responses and protectivity of these vaccines have varied widely, and no single approach has shown safety and efficacy for all coronaviruses.

For FIPV, live-attenuated, inactivated, and subunit vaccines based on recombinant or purified spike protein, have not only failed to protect against FIPV disease, but have resulted in immune enhancement of infection and disease, a response disturbingly reminiscent of the result following vaccination of humans with inactivated vaccines for measles and respiratory syncytial virus. The most useful animal coronavirus vaccine has been the live-attenuated vaccine for IBV. However, efficacy is still clearly less than optimal. In addition, reversion to virulence may occur, and recombination of the vaccine strain with wild-type viruses has occurred, with disease in chickens caused by the recombinant vaccine-wild-type viruses.

For the most part, vaccines have not been pursued in the past for human coronaviruses, likely because the frequency and severity of infections could not be well defined, and the determinants for protection have not been identified. It is also known that 229E and OC43 can reinfect humans, possibly as often as every other year, suggesting that vaccine strategies may need to be targeted toward limitation of disease severity, since prevention may not be possible.

Together, the known biological properties of coronaviruses, as well as the concerns with limited protection or immune enhancement of disease by coronavirus vaccines, are compelling arguments for a new approach in the development of live, attenuated vaccines that are less subject to reversion and recombination, but possess normal pathways for infection and immune response. This need is all the more critical in light of the emerging human SARS situation.

### SUMMARY OF THE INVENTION

Thus, in accordance with the present invention, there is provided a live, attenuated virus of the order Nidovirales, said virus characterized as comprising a genome encoding a replicase polyprotein comprising at least one proteinase cleavage with mutations that exhibit normal, reduced or no cleavage. The virus may be of the family Coronaviridae, such as a coronavirus or a torovirus. Particular coronaviruses are avian infectious bronchitis virus, bovine coronavirus, canine coronavirus, feline infectious peritonitis virus, human coronavirus 229E, human coronavirus OC43, murine hepatitis virus, porcine epidemic diarrhea virus, porcine hemagglutinating encephalomyelitis virus, porcine transmissible gastroenteritis virus, rat coronavirus, turkey coronavirus, severe acute respiratory syndrome virus, or rabbit coronavirus. Toroviruses include Berne virus or Breda virus. The virus may also be of the family Arteriviridae, such as arteriviruses like equine arteritis virus, Lelystad virus or simian hemorrhagic fever virus.

The cleavage site may be a C1-C14 cleavage site. In particular, the cleavage site may be a murine hepatitis virus p28-p65 (C1), p65-p210 (C2), or p59-p42 (C13) cleavage site, or a SARS-CoV nsp1-nsp2 (C1), nsp2-nsp3 (C2) or nsp14-nsp15 (C13) cleavage site. The cleavage site may contain an amino acid deletion, an amino acid insertion or an amino acid substitution. Alternatively, cleavage site may be wild-type, but cleavage is reduced or eliminated by an allosteric mutation in the proteinase(s) responsible for cleavage. The replicase polyprotein may comprise at least a second proteinase cleavage site with changes resulting in normal reduced, or no cleavage as compared to wild-type virus cleavage.

In another embodiment, there is provided a method of inducing an anti-viral immune response in a host comprising administering to said host a live, attenuated vaccine of the order Nidovirales, said vaccine characterized as comprising a genome encoding a replicase polyprotein comprising at least one proteinase cleavage site with changes resulting in normal, reduced, or no cleavage. The vaccine may be of the family Coronaviridae, such as a coronavirus or a torovirus. Particular coronaviruses are avian infectious bronchitis virus, bovine coronavirus, canine coronavirus, feline infectious peritonitis virus, human coronavirus 229E, human coronavirus OC43, murine hepatitis virus, porcine epidemic diarrhea virus, porcine hemagglutinating encephalomyelitis virus, porcine transmissible gastroenteritis virus, rat coronavirus, turkey coronavirus, severe acute respiratory syndrome virus, or rabbit coronavirus. Additional coronaviruses include HcoV-NL63 and HcoV-NL Toroviruses include Berne virus or Breda virus. The vaccine may also be of the family Arteriviridae, such as arteriviruses like equine arteritis virus, Lelystad virus or simian hemorrhagic fever virus.

The cleavage site may be selected from the C1, C2, C3, C4, C5, C6, C7, C8, C9, 10, C11, C12, C13 and C14 cleavage sites, with C1 representing the first recognized cleavage site in the polyprotein. In particular, the cleavage site may be a murine hepatitis virus p28-p65 (C1), p65-p210 (C2), or p59-p42 (C13) cleavage site, or a SARS-CoV nsp1-nsp2 (C1), nsp2-nsp3 (C2) or nsp14-nsp15 (C13) cleavage site. The cleavage site may contain an amino acid deletion, an amino acid insertion or an amino acid substitution. Alternatively, cleavage site may be wild-type, but cleavage is reduced or eliminated by an allosteric mutation in the proteinase(s) responsible for cleavage. The replicase polyprotein may comprise at least a second proteinase cleavage site with changes resulting in normal, reduced, or no cleavage as compared to wild-type virus cleavage. The vaccine may be administered intramuscularly, subcutaneously, intradermally, intranasally, or orally. The method may further comprise administering an immunostimulant such as an adjuvant like alum, MF-59, QS-21, or others, or a biologic immunomodulatory molecule such as a cytokine, immunological receptor, or antibody. The host may be a dog, a cow, a pig, a cat, a mouse, a rat, a horse, a chicken, a turkey, a monkey, a ferret, or a human.

In another embodiment, there is provided a live-attenuated virus of the order Nidovirales for safe laboratory use and as a platform for safe growth of high titer virus for use in inactivated virus vaccines. Said virus is characterized as comprising a genome encoding a replicase polyprotein comprising at least one proteinase cleavage site with changes resulting in normal, reduced, or no cleavage. The virus may be of the family Coronaviridae, such as a coronavirus or a torovirus. Particular coronaviruses are avian infectious bronchitis virus, bovine coronavirus, canine coronavirus, feline infectious peritonitis virus, human coronavirus 229E, human coronavirus OC43, murine hepatitis virus, porcine epidemic diarrhea virus, porcine hemagglutinating encephalomyelitis virus, porcine transmissible gastroenteritis virus, rat coronavirus, turkey coronavirus, severe acute respiratory syndrome virus, or rabbit coronavirus. Additional coronaviruses include HcoV-NL63 and HcoV-NL. Toroviruses include Berne virus or Breda virus. The virus may also be of the family Arteriviridae, such as arteriviruses like equine arteritis virus, Lelystad virus or simian hemorrhagic fever virus.

The cleavage site may be selected from the C1, C2, C3, C4, C5, C6, C7, C8, C9, 10, C11, C12, C13 and C14 cleavage sites, with C1 representing the first recognized cleavage site in the polyprotein. In particular, the cleavage site may be a murine hepatitis virus p28-p65 (C1), p65-p210 (C2), or p59-p42 (C13) cleavage site, or a SARS-CoV nsp1-nsp2 (C1), nsp2-nsp3 (C2) or nsp14-nsp15 (C13) cleavage site. The cleavage site may contain an amino acid deletion, an amino acid insertion or an amino acid substitution. Alternatively, cleavage site may be wild-type, but cleavage is reduced or eliminated by an allosteric mutation in the proteinase(s) responsible for cleavage. The replicase polyprotein may comprise at least a second proteinase cleavage site with changes resulting in normal, reduced, or no cleavage as compared to wild-type virus cleavage. The virus may be grown to high titer and inactivated by formalin, heat, irradiation, or a combination of these approaches. The inactivated virus vaccine may be administered intramuscularly, subcutaneously, intradermally, intranasally, or orally. The method may further comprise administering an immunostimulant such as an adjuvant like alum, MF-59, QS-21, or others, or a biologic immunomodulatory molecule such as a cytokine, immunological receptor, or antibody. The host may be a dog, a cow, a pig, a cat, a mouse, a rat, a horse, a chicken, a turkey, a monkey, a ferret, or a human.

In yet another embodiment, there is provided a nidovirus genome, said genome encoding a replicase polyprotein comprising at least one proteinase cleavage site with changes resulting in normal, reduced, or no cleavage. Also provided is a nidovirus replicase polyprotein comprising at least one proteinase cleavage site with changes resulting in normal, reduced or no cleavage.

In still yet another embodiment, there is provided a vaccine comprising (a) a live-attenuated virus of the order Nidovirales, said virus characterized as comprising a genome encoding a replicase polyprotein comprising at least one proteinase cleavage site with changes resulting in normal, reduced or no cleavage, and (b) a pharmaceutically acceptable diluent. The vaccine may be formulated as a unit dose of 10⁶ to 10¹⁴ infectious particles, and may be formulated to be dispensed as unit doses of 0.1 ml to 1.0 ml. The vaccine may further comprise a preservative. The vaccine may be lyophilized.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-C—Coronavirus Genome Organization, Replicase Protein Expression and Processing and Replicase Polyprotein Cleavage Sites. The coronavirus genome is 29 to 32 kilobases in length. The replicase gene (gene 1) of SARS-CoV (FIG. 1A) and MHV-A59 (FIG. 1B) is translated from input genome RNA to yield the ORF 1a polyprotein or the ORF 1a/1b polyprotein (lab shown here). Structural (S—spike; E—envelope, M—membrane, and N—nucleocapsid) and accessory genes are expressed from a 3′ nested set of subgenomic mRNAs (not shown). Confirmed or predicted mature replicase gene products are shown in panel (FIG. 1C). Limits of protein domains are indicated by vertical bars, by nsp #, by predicted mass (kDa) and by function if confirmed or predicted: PLP1 and PLP2-papain-like proteinase 1 and 2; 3C—3C-like proteinase; pol—putative RNA dependent RNA polymerase; hel—NTPase/helicase; MP1 and MP2—membrane spanning proteins; ExoN—putative 3′ to 5′ exonuclease; XendoU—putative poly(U)-specific endoribonuclease; O-MT-2′-O-methyltransferase. In the middle of figure the likely intermediate and mature protein products of proteolytic processing are shown. The cleavage sites (CS) 1-14 are indicated.

FIG. 2-Organization and comparison of N terminal regions of the MHV and SARS-CoV replicase polyproteins. The schematics of MHV and SARS-CoV N-terminal 3600 amino acids are shown. The size and location of nsp1, nsp2, and nsp3 are indicated by rectangles. The nsp3 domain is shown in grey. The PLP domains of nsp3 are shown as small boxes; papain-like proteinases 1 and 2 (PLP1, PLP2). 3CLpro domain is indicated but not cleavage sites. Known cleavage sites (CS1, CS2, and CS3) in MHV are indicated. Known cleavages are illustrated by solid lines/arrows. Predicted or possible cleavages are shown as dashed lines/arrows. For SARS-CoV, predicted proteins and cleavage sites are shown, based on sequence analysis. Since by sequence analysis only an ortholog of PLP2 is present in SARS-CoV, it is predicted to cleave at CS1 and CS2 in addition to CS3.

FIG. 3—General strategy for reverse genetic cloning mutagenesis and recovery of virus mutants for MHV and SARS-CoV. Purified genome RNA is used to derive cDNA cloned fragments of the genome (fragments A-G for MHV and fragments A-F for SARS-CoV. Mutagenesis occurs to individual fragments (stars). Genome fragments are excised by restriction enzymes, and the purified fragments are ligated in vitro in a unidirectional manner. In vitro transcription of mutant genome plus-strand RNA is driven off T7 promoter (black arrowhead), and full-length infectious RNA is electroporated into cells with recovery of mutant virus from the cell media supernatant.

FIGS. 4A-E—Mutagenesis and characterization of mouse hepatitis virus (MHV) CS1 mutants. FIG. 4A. Schematic of replicase polyprotein with location of CS1 between nsp1 and nsp2 (p28-p65). The P3 through P3′ residues are shown, with cleavage between Gly_Val. Amino acid residues substituted (H-His, V-Val, A-Ala) or deleted (del) are indicated. Cleavage in vitro and recovery of virus is indicated. FIG. 4B. CS1 processing in mutant viruses. Cells were infected for 6 h, labeled with [³⁵S]met/cys, lysed, IP'd with UP102 (αp28, αp65, and analyzed on a gel. Markers are to the left and the location of p28, p65, and uncleaved p28-p65 (p93) are indicated to the right. Wt—wild-type A59; icwt—assembled wild-type, mut 8,9,3,4,5, ΔCS1 as in FIG. 4A. FIG. 4C. Virus growth. Virus stocks were used to infect DBT cells and supernatant virus was measured by plaque assay. FIG. 4D. Viral RNA synthesis. Infected DBT cells were metabolically labeled with [³H]-uridine in the presence of Actinomycin D from 6-7 h p.i. TCA precipitable incorporation of [³H]-uridine was measured. Viruses are labeled as in FIG. 4A. FIG. 4E. Localization of uncleaved p93. Infected DBT cells were fixed with methanol at 6 h p.i. and stained with antibodies against: top row—p28 (green) and N (red) or bottom row—p93 (green) and N (red). Colocalized pixels are in yellow. Note almost complete p28/N colocalization but significant p93 that is not colocalized with N.

FIGS. 5A-C—Mutations at MHV CS2 and papain-like proteinase 1 (PLP1). FIG. 5A. Schematic of nsp1-nsp3 region of the MHV replicase polyprotein, showing location of nsp1 (p28), nsp2 (p65) and nsp3 (p21) with CS1, CS2, and PLP domains indicated. FIG. 5B. Mutations of CS2. Cells were infected with recovered virus mutants and immunoprecipitation of radiolabeled proteins was performed with α-nsp1 and α-nsp2. Mock: mock infected cells; A59: wild-type lab MHV; Mutants as indicated for P2-Cys₈₃₁ and P1-Ala₈₃₂. Δ₈₃₁₋₈₃₃ indicates deletion mutant of P2-P1′. FIG. 5C. Mutations of PLP1. Catalytic Cys₁₁₂₁Gly mutant was recovered seven days post-electroporation and used to infect cells that were immunoprecipitated for nsp1-p28 and nsp 2-p65. Markers are to the left and proteins are indicated.

FIG. 6-Mutations in SARS-CoV replicase polyprotein cleavage sites. Nsp1-4 of the polyprotein is shown at top. CS1A (probable cleavage), CS1B (possible alternative CS matching MHV CS1), and probable CS2 and CS3 indicated. Sequences below indicate introduced deletions of residues or mutations. All mutations have been cloned in SARS-CoV genome fragment A, B, or C and have been sequence confirmed.

FIG. 7-Location and sequence of SARS-CoV 3CLpro cleavage sites. Schematic of 1a/b polyprotein shows PLP and 3CLpro cleavage sites, with location of CS11, 12, and 13 indicated. Amino acid sequence flanking cleavage sites is shown, with LQ_(S,A) indicating cleavage between Q and S or A.

### DESCRIPTION OF ILLUSTRATIVE EMBODIMENTS

### I. The Present Invention

Continued replicase protein expression and processing is required for ongoing viral RNA synthesis and productive Nidovirus infection. In addition, studies suggest roles for replicase proteins in the formation and function of replication complexes, virus assembly, and pathogenesis. The functional proteins may be activated, inactivated, or altered in function by proteolytic processing. The description of several stable intermediate precursors from the replicase polyprotein further suggests that proteolytic maturation may regulate both the availability and the activity of replicase proteins. As such, proteolytic cleavage sites may represent independent genetic elements that are critical to all processes of viral replication and pathogenesis. Further, the cleavage sites and cleavage events are well-defined genetic targets for inhibition of replication or disease. However, nothing is known of the role of these genetic elements in coronavirus replication.

The present inventor has used the murine coronavirus, mouse hepatitis virus (MHV), to demonstrate the feasibility of developing attenuated Nidoviruses with altered replicase proteins for use as vaccines and as platforms for safe growth. MHV is a well-developed model for understanding the replication, pathogenesis, and immune determinants of coronavirus infections and disease. In addition, MHV has been an important virus model for demyelinating encephalititides, specifically multiple sclerosis. In this study, the inventor used MHV to determine if mutations in cleavage sites in the coronavirus replicase gene could yield stably attenuated mutants.

Specifically, the inventor initially tested the hypothesis that the first cleavage event in the MHV replicase polyprotein, between p28 and p65 (CS1), was required for viral replication. Surprisingly, both mutants that maintained cleavage and those that abolished cleavage allowed recovery of viable virus. Various degrees of growth were observed, ranging from cleaving mutants with wild-type growth in culture, to non-cleaving mutations and deletions with significantly impaired growth. Mutations and deletions at CS2, CS13, and CS1+CS2 also were recovered in viable virus. This result led to the concept of using such mutants as stably attenuated vaccines with distinct advantages. First, no proteins corresponding to the replicase proteins have been identified in virions, nor do they appear to be specifically targeted to the cell surface. Thus, there would be expected to be less immune pressure for reversion during infection. Second, other deletions or alterations in the proteins themselves appear to be lethal for replication, so that it may be more difficult for the virus to introduce second site compensating mutations. And third, cleavage knockout mutants that delete P2, P1 and P1′ still permit recovery of viable virus that grows to titers of greater than 10⁶ infectious particles per ml. Thus, the tolerance for cleavage site deletion can be exploited to create virus with even less likelihood of reversion to virulence.

Clearly, the primary concern in using attenuated vaccines is the possibility of reversion to virulence by mutations or recombination. And it certainly has been well demonstrated that coronaviruses have a variety of mechanisms, as well as the capacity, to recover from seemingly insurmountable impairments such as dramatic rearrangements and deletions of genes downstream from the replicase. The high rates of mutation and homologous recombination all provide mechanisms to recover virulence. However, in absence of recombination during coinfection, the likelihood of regeneration of a cleavage site deletion would be very unlikely, particularly since the replication defect, while significant, is not lethal. Further, the results reported here suggest the possibility that the virus may tolerate alterations of other replicase polyprotein cleavages as well as combinations of cleavage site mutations.

For human viruses acquired by the respiratory route, live-attenuated vaccines with alterations in protein processing would have several potential advantages. Because there is no alteration in the viral structural glycoproteins, it is predicted that replicase protein cleavage mutants would have normal “wild-type” transmission, tropism, attachment, entry and uncoating, and thus could theoretically be administered by oral, intranasal or inhaled approaches. The initial replication and spread from the respiratory epithelium and lymphoid organs might allow for the development of both systemic and mucosal immunity.

Studies with other animal coronavirus vaccines suggest that viral replication may be critical for protection from virus challenges. The use of a virus stably attenuated in replication also avoids concerns about atypical infections with wild-type viruses following vaccination with inactivated viruses or purified viral proteins, such as occurred with measles virus and respiratory syncytial virus, and also seen with the vaccines for the feline coronavirus, FIPV. Most importantly, the use of a live-attenuated virus allows for both humoral and cellular immunity.

Thus, as a general strategy for nidovirus vaccines, the inhibition of polyprotein processing is widely applicable to viruses with significantly different hosts, virulence, pathogenesis and disease. All known nidoviruses use a similar strategy to translate and process their replicase proteins from a polyprotein. The similarities in the viral proteinases, proteins and cleavage sites, particularly in the group 2 coronaviruses, indicate that similar strategies could be used to develop other attenuated virus vaccine strains. Importantly, in the context of the newly emerged human SARS coronavirus, the cleavage sites and putative replicase proteins have been predicted to be highly conserved with the group 2 coronaviruses.

### II. Order Nidovirales

Nidoviruses are positive-stranded RNA viruses infect a wide range of vertebrates. The virions are enveloped, pleomorphic, spherical, or kidney-shaped. Surface projections of envelope distinct; club-shaped; dispersed evenly over all the surface. Two families are established: Family Arteriviridae and Family Coronaviridae.

A. Coronaviridae

This virus infects host in the Domain Eucarya, Kingdom Animalia, Phylum Chordata, Subphylum Vertebrata, Classes Mammalia and Aves, Orders Primates, Carnivora, Perissodactyla, Artiodactyla, Rodentia, and Lagomorphia. It is not known to be transmitted by means not involving any vector. World-wide distribution is likely.

Virions are enveloped, slightly pleomorphic, spherical or kidney shaped, and about 120-160 nm in diameter. Surface projections of envelope are distinct, club-shaped, spaced widely apart and dispersed evenly over all the surface. Nucleocapsids are rod-shaped (straight or bent), about 9-13 nm in diameter. Virion-associated RNA nucleocapsids exhibit helical or tubular symmetry.

Molecular mass (Mr) of the virion 400×106. Buoyant density is 1.23-1.24 g cm-3 in CsCl, and 1.15-1.19 g cm-3 in sucrose. The sedimentation coefficient is 300-500S. Under in vitro conditions, virions are stable in acid environment (pH 3), relatively stable in presence of Mg⁺⁺. Virions are sensitive to heat, lipid solvents, non-ionic detergents, formaldehyde, and oxidizing agents.

Virions contain one molecule of linear positive-sense single stranded RNA with a total genome length is 20,000-33,000 nt. The 5′ end of the genome has a cap, and the 3′ end has a poly(A) tract. Subgenomic mRNA is found in infected cells.

Five structural virion proteins found ranging is size between 18,0000 and 220,000 Da. The first is the surface glycoprotein or spike (S) protein. The S protein is responsible for attachment to cells, hemagglutination and membrane fusion. It has a carboxy-terminal half with a coiled-coil structure. The second largest protein (30,000-35,000 Da) is the integral membrane protein (M) which spans the virus envelope three times, with only 10% protruding at the virion surface. The third largest protein (50,000-60,000 Da) is the nucleocapsid protein (N). The fourth largest protein (65,000 Da) is the hemagglutinin-esterase protein (HE), which forms short surface projections, and can have receptor binding, hemagglutination and receptor destroying activities. The fifth largest protein (10,000-12,000 Da) is tentatively designated as the small membrane protein (sM), detected in avian infectious bronchitis virus (IBV) and porcine transmissible gastroenteritis virus (TGEV).

The virus exhibits distinct antigen determinants on envelope and spikes, those corresponding to each of the major structural glycoproteins—S, HE, M, and N. Antigenic specificity of virion can be determined by neutralization tests (S and HE), or complement fixation tests (M). Protective immunity is induced in form of complement independent neutralizing antibodies.

The Coronaviridae family is split into two groups—coronavirus and torovirus. Coronaviruses include avian infectious bronchitis virus, bovine coronavirus, canine coronavirus, feline infectious peritonitis virus, human coronavirus 229E, human coronavirus OC43, murine hepatitis virus, porcine epidemic diarrhea virus, porcine hemagglutinating encephalomyelitis virus, porcine transmissible gastroenteritis virus, rat coronavirus, turkey coronavirus, severe acute respiratory syndrome virus, rabbit coronavirus, or the recently identified SARS associated human coronavirus or human coronaviruses NL and NL63. Toroviruses include Berne virus or Breda virus.

B. Arterivirus

The family Arterivirus infects hosts of the Domain Eucarya, Kingdom Animalia, Phylum Chordata, Subphylum Vertebrata, Class Mammalia, Orders Primates, Perissodactyla, Artiodactyla, and Rodentia, and is transmitted by means not involving a vector. Serological relationships between different members are not detectable, i.e., cross-reactivity not found.

Virions are enveloped, spherical, and about 60 nm in diameter. Surface projections on envelope not present, but honeycomb-like subunits of 10-14 nm have been observed. Nucleocapsids are visometric and about 35 nm in diameter. The virion RNA-nucleocapsid exhibits icoshedral symmetry. Surface projections on nucleocapsid are small dispersed over the entire surface.

Buoyant density is 1.17-1.2 g cm-3 in CsCl, and 1.13-1.17 g cm-3 in sucrose. The sedimentation coefficient 200-230S.

Virions contain one molecule of linear single stranded RNA. The total genome length is 13000 nt. The 5′ end of the genome has a cap (cap sequence m7G5 ppp5′GmpNp). The 3′ end has a poly(A) tract that is about 50 nucleotides long. Each virion contains longer than full length copy.

At least four structural virion proteins are found. Protein sizes range from 30,000 to 42,000 Da. The first is an N-glycosylated surface protein designated GL, which is about 25,000 Da. The second is an N-glycosylated surface protein designated GD, which is about 16,000 Da. The third is a non-glycosylated triple membrane spanning integral membrane protein designated M. The fourth is a 12,000 Da nucleocapsid protein, designated N, also non-glycosylated. Virions contain 10% lipid (envelope).

Selected Arteriviruses include equine arteritis virus, Lelystad virus and simian hemorrhagic fever virus.

### III. Replicase Polyprotein

A. Gene and Protein Structure

The coronavirus replicase gene (also known as gene 1 or the polymerase gene) comprises 22 kB of the coronavirus genome, corresponding to some 7800 amino acids, and is composed of two overlapping open reading frames—ORE1a and 1b. Following uncoating of the RNA genome in the cell cytoplasm, the replicase gene is translated as either an ORF 1a polyprotein (495 kD) or as an ORF1ab fusion polyprotein (803 kD), with translation of ORF 1b requiring a ribosomal frameshift event at the end of ORF 1a. The intact replicase polyproteins are not detected during natural infection, since maturation proteolytic cleavages occur cotranslationally by three proteinase functions encoded in ORF 1a polyprotein. The proteolytic processing results in 15 mature proteins, including the proteinases, an RNA helicase, and a putative RNA-dependent RNA polymerase. The MHV proteins are set forth in Table 1, and a schematic comparing MHV replicase polyprotein with a SARS strain replicase polyprotein is given in FIG. 8.

TABLE 1
MHV and SARS-CoV Replicase Protein Domains and Mature Proteins

| MHV Protein | SARS Protein | |
|---|---|---|
| Designation | Deignation | Function |
| p28 | nsp1-p20 | Unknown; localizes to replication complexes early in infection where it |
| aa1-247 | | associates with membranes by easily disrupted peripheral mechanisms |
| p65 | nsp2-p70 | Unknown; shown to associate throughout infection with membranes of |
| aa248-834 | | replication complexes at sites of viral RNA synthesis, likely by |
| | | interactions with other proteins |
| p210 | nsp3-p213 | Encodes a protein with one or two papain-like proteinase domains (PLP) |
| aa833-2837 | | that cleave the first three (C1-C3) cleavage sites |
| MP1 | nsp4-p56-HD1 | Highly hydrophobic, membrane associated, found in replication |
| 2837- | | complexes |
| 3CLpro | nsp5-p33 | Picornain like proteinase responsible for cleavage at C4-C14 |
| MP2 | nsp6-p32 | Highly hydrophobic, membrane associated, localization in cells |
| | HD2 | unknown |
| p10 | nsp7 p10 | Associates with p22, p12, p15 in replication complexes |
| p22 | nsp8-p22 | Associates with p10, p12, p15 in replication complexes |
| p12 | nsp9-p12 | Associates with p22, p10, p15 in replication complexes |
| p15 | nsp10-p15 | Associates with p22, p12, p10 in replication complexes. May play role |
| | | in RNA synthesis |
| nsp11? | nsp11? | Predicted—no protein identified |
| Polymerase | nsp12-pol | Putative RNA dependent RNA polymerase, localizes to replication |
| (pol, p100) | | complexes |
| Helicase (hel, | nsp13-p67-hel/ | RNA unwinding and NTPase activities |
| p67) | ATPase | |
| p57 | nsp14-p59- | Predicted 3′ to 5′ Exoribonuclease |
| | ExoN | |
| p42 | nsp15-p42- | Predicted PolyU specific endoribonuclease |
| | XendoU | |
| p33 | nsp16-p33-O- | Predicted 2 O-methyltransferase |
| | MT | |

Proteins from the replicase gene are proposed to comprise the necessary and sufficient viral proteins to direct all stages of MHV mRNA synthesis and genome replication. In addition, it has been shown that inhibition of polyprotein processing at any time during infection by chemical inhibitors results in rapid shutoff of viral RNA synthesis, indicating that the general process of proteolytic maturation is required for RNA synthesis and viral growth. Although there are differences in mature replicase proteins among different coronaviruses, particularly in the amino-terminal 100 kD of the polyproteins, the general strategy of proteinase cleavage sites, proteinases and products is maintained. More specifically, proposed critical functional proteins (polymerase, helicase, proteinase) are highly conserved across different coronaviruses. Notably, the new SARS associated coronaviruses appears by sequence analysis to use the same of protein organization and cleavage as MHV.

B. Cleavage Sites

By convention, the present invention identifies the polyprotein cleavage sites as C1-C14. These sites are defined as cleaving between adjacent products. Examples for MHV and SARS-CoV are set forth in Table 2, below.

TABLE 2
MHV and SARS-CoV Replicase Polyprotein Cleavage Sites

| | Upstream Protein MHV | Downstream Protein MHV |
|---|---|---|
| Cleavage Site | (SARS-CoV) | (SARS-CoV) |
| C1 | p28 (nsp1) | p65 (nsp2) |
| C2 | p65 (nsp2) | p210 (nsp3) |
| C3 | p210 (nsp3) | MP1 (nsp4) |
| C4 | MP1 (nsp4) | 3Clpro (nsp5) |
| C5 | 3Clpro (nsp5) | MP2 (nsp6) |
| C6 | MP2 (nsp7) | p10 (nsp8) |
| C7 | p10 (nsp8) | p22 (nsp9) |
| C8 | p22 (nsp9) | p12 (nsp10) |
| C9 | p12 (nsp10) | p15 (nsp11) |
| C10 | p15 (nsp11) | Polymerase (nsp12) |
| C11 | Polymerase (nsp12) | Helicase (nsp13) |
| C12 | Helicase (nsp13) | P59 (nsp14-ExoN) |
| C13 | p57 (nsp14) | p42 (nsp15-XendoU) |
| C14 | p42 (nsp15) | p33 (nsp16-O-MT) |

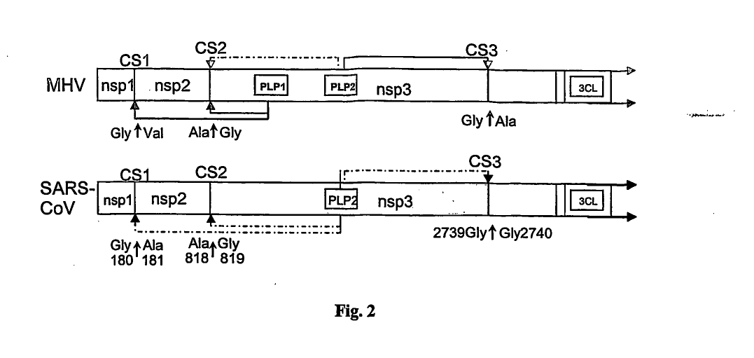

The first two proteins, p28 (nsp1) and p65 (nsp2) have unknown functions but are likely involved both in RNA synthesis and transitions from RNA synthesis to virus particle assembly. The third protein processed from the replicase polyprotein is p210 (Schiller et al., 1998). The MHV p210 protein (nsp3 of SARS-CoV) has a predicted mass of 221 kD, and contains the two papain-like proteinase domains (PLP1 and PLP2) that have been shown to cleave the first three cleavage sites (CS1, CS2 and CS3) at the carboxy-termini of p28, p65 and p210, respectively. The apparent difference between coronaviruses in the predicted number of proteinases, and the differences in the size and number of proteins in the amino-terminal half of the polyprotein, was interpreted to indicate a lack of common critical functions in this region of gene. A recent study used sequence comparisons, parsimony analyses, and studies of the cleavage sites and proteinase functions to compare the coronavirus p210 and the corresponding p195 proteins of the human coronavirus 229E (HCoV-229E) and infectious bronchitis virus (IBV) (Ziebuhr et al., 2001). The analyses identified common domains of the coronavirus p210/p195 proteins (FIG. 2), several of which had previously been predicted or conformed for MHV (Lee et al., 1991). The amino-terminal domain of p210 was referred to as the “acidic domain” (Ac) based on the concentration of acidic residues. The PLP1 domain consists of the sequence required for proteinase activity during in vitro cleavage reactions (Bonilla et al. 1995). The X domain is a region of increased conservation among the different coronavirus p210/p195 proteins with no known or predicted functions (Lee et al., 1991). The functional PLP2 domains are a variable distance from the X domains, and have been less completely characterized as to their functional requirements. Both PLP1 and PLP2 have been demonstrated to function with a catalytic dyad of Cys and His residues (Baker et al., 1993; Bonilla et al., 1995; Kanjanahaluethai and Baker, 2000). Finally, a Y domain consists of a region incorporating two stretches of predominantly hydrophobic residues that predict membrane-spanning helices (Lee et al., 1991).

Coronavirus PLPs have a zinc finger motif in the predicted papain-like fold of the enzymes, with predicted similarities to the human transcription elongation factor TFIIS (Herold et al., 1999). The zinc finger has been shown to bind zinc, which is required for PLP function in vitro. Mutations in this motif abolish proteolytic activity. It has been suggested based on these features and demonstrated contributions of the zinc finger to RNA synthesis in the arterivirus, equine arteritis virus (EAV) (Tijms et al., 2001), that the zinc finger may serve functions in addition to PLP proteolytic activity.

Studies of PLP1 and PLP2, as well as identification and detailed mutagenesis of replicase polyprotein cleavage sites, have been performed in vitro. PLP1 has been shown to proteolytically process the first two cleavage sites in the MHV replicase polyprotein: between p28 and p65 at 247G/V248 (referred to as CS1) and between p65 and p210 at 832A/G833 (CS2) (Dong and Baker, 1994; Hughes et al., 1995; Bonilla et al., 1997; Baker et al., 1993). PLP2 has been shown to cleave at the carboxy-terminus of p210 (CS3), likely in a cis autocat (Kanjanahaluethai and Baker, 2000; Kanjanahaluethai et al., 2001). Although the MHV CS3 cleavage site has not been reported, by direct comparison with identified IBV PLP2 cleavage site the MHV-A59 p210 carboxy-terminal cleavage (CS3) would be predicted to be 2837G/A2838. Analysis of the MHV CS1 and CS2 in comparison with other group 1 coronaviruses (TGEV, HCoV-229E) (Elcouet et al., 1995; Herold et al., 1993), group 2 coronaviruses MHV-JHM, BCV) (Yoo and Pei, 2001; Chouljenko et al., 2001), and group 3 coronaviruses (IBV) (Boursnell et al., 1987), has demonstrated similarities at the P1/P1′ cleavage dipeptides; Gly or Ala at P1 of all coronavirus PLP CS, and Val, Ala or Gly at P1′. HCoV is the exception, using Asn in the P1′ position. Overall, P5, P2, P1 and P1′ have been most intolerant of changes, with mutations at these sites disrupting cleavage in vitro.

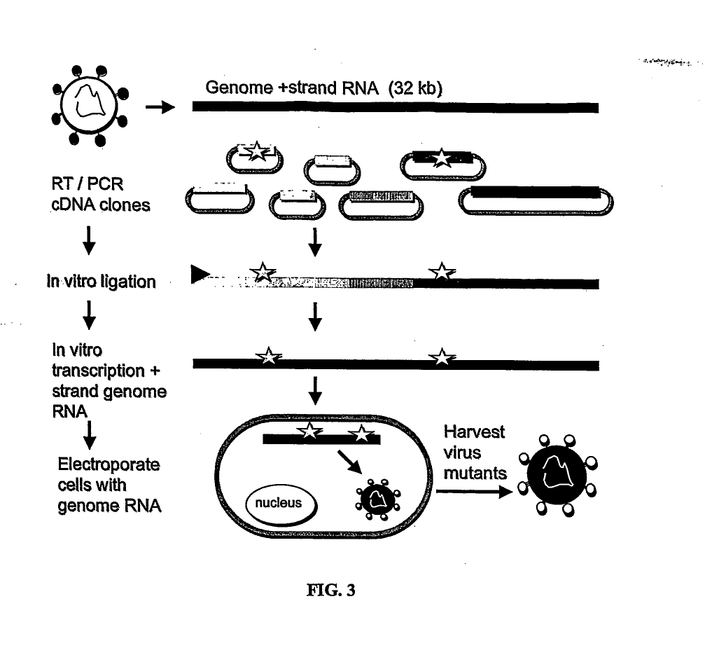

Analysis of the coronavirus PLPs and their cognate cleavage sites suggests that PLP1 and PLP2 are paralogous proteinases, originating from a common coronavirus or pre-coronavirus ancestor, and that they have diverged over time (Ziebuhr et al., 2001) (FIG. 3). For example, all coronaviruses except IBV and SARS-CoV express both PLP1 and PLP2 activities and share the common feature that PLP1 cleaves CS1 and CS2. IBV only expresses a PLP2 that cleaves at a single site equivalent to CS2. In IBV, a functional PLP1 is not detected, whereas a residual, highly altered and inactive PLP1 domain has recently been identified by sequence comparison (Ziebuhr et al., 2001). These observations have led to the hypothesis that there may be overlap of cleavage site specificity and PLP activity, and possible redundancy of cleavage activity, with PLP 2 able to mediate cleavages at PLP1 cognate sites. This has been demonstrated to be true for HCoV, with both PLP1 and PLP2 able to cleave CS2 in vitro (Ziebuhr et al., 2001). In fact, the data suggest that the “normal” CS2 cleavage event may involve the cooperative activity of PLP1 and PLP2. However, it was also demonstrated that when PLP1 was catalytically inactivated, PLP2 was able to independently mediate CS2 cleavage in vitro. This conclusion is strengthened by the finding of only a putative active PLP2 domain in SARS-CoV and the demonstration that CS1 and CS2 can be cleaved by this PLP2.

### IV. Engineering of Nidovirus Genomes

Thus, in accordance with the present invention, it will be desirable to create a variety of different cleavage mutants in Nidovirus replicase polyproteins. Mutagenesis is the process whereby changes occur in the structure of a genome. Mutation can involve modification of the nucleotide sequence of a single gene, blocks of genes or a whole chromosome. Changes in single genes may be the consequence of point mutations which involve the removal, addition or substitution of a single nucleotide base within a DNA sequence, or they may be the consequence of changes involving the insertion or deletion of large numbers of nucleotides.

Mutations can arise spontaneously as a result of events such as errors in the fidelity of DNA replication or the movement of transposable genetic elements (transposons) within the genome. They also are induced following exposure to chemical or physical mutagens. Such mutation-inducing agents include ionizing radiations, ultraviolet light and a diverse array of chemical such as alkylating agents and polycyclic aromatic hydrocarbons all of which are capable of interacting either directly or indirectly (generally following some metabolic biotransformations) with nucleic acids. The DNA lesions induced by such environmental agents may lead to modifications of base sequence when the affected DNA is replicated or repaired and thus to a mutation. Mutation also can be site-directed through the use of particular targeting methods. Any number of different mutagenic approaches may be taken, as described below.

A. Nidovirus Genomes

One of skill in the art may use various Nidovirus replicase sequences to design specific mutations that abolish or reduce replicase protein cleavage. The following constitute non-limiting examples of Nidovirus genomic accession nos., each of which are incorporated by reference: human coronavirus 229E (NC002645), SARS TOR2 (AY274119), SARS HKU-39849 (AY278491), SARS CUHK-W1 (AY278554), bovine coronavirus (BCV) (NC003045), avian infectious bronchitis virus (IBV) (NC001451), transmissible gastroenteritis virus (TGEV), (NC002306), mouse hepatitis virus (MHV) (NC001846).

B. Random Mutagenesis

In one embodiment, random mutagenesis may be applied. This will, of course, require an additional step of screening for the desired mutations. Screening will typically be accomplished by nucleic acid hybridization (Southern or Northern blotting), sequencing, or SnP analysis, methods of which are well known to those of skill in the art.

i) Insertional Mutagenesis

Insertional mutagenesis is based on the inactivation of a gene via insertion of a known DNA fragment. Because it involves the insertion of some type of DNA fragment, the mutations generated are generally loss-of-function, rather than gain-of-function mutations. However, there are several examples of insertions generating gain-of-function mutations (Oppenheimer et al. 1991). Insertion mutagenesis has been very successful in bacteria and Drosophila (Cooley et al. 1988) and recently has become a powerful tool in corn (Schmidt et al. 1987); Arabidopsis; (Marks et al., 1991; Koncz et al. 1990); and Antirrhinum (Sommer et al. 1990).

Transposable genetic elements are DNA sequences that can move (transpose) from one place to another in the genome of a cell. The first transposable elements to be recognized were the Activator/Dissociation elements of Zea mays (McClintock, 1957). Since then, they have been identified in a wide range of organisms, both prokaryotic and eukaryotic.

Transposable elements in the genome are characterized by being flanked by direct repeats of a short sequence of DNA that has been duplicated during transposition and is called a target site duplication. Virtually all transposable elements whatever their type, and mechanism of transposition, make such duplications at the site of their insertion. In some cases the number of bases duplicated is constant, in other cases it may vary with each transposition event. Most transposable elements have inverted repeat sequences at their termini. These terminal inverted repeats may be anything from a few bases to a few hundred bases long and in many cases they are known to be necessary for transposition.

Prokaryotic transposable elements have been most studied in E. coli and Gram negative bacteria, but also are present in Gram positive bacteria. They are generally termed insertion sequences if they are less than about 2 kB long, or transposons if they are longer. Bacteriophages such as mu and D108, which replicate by transposition, make up a third type of transposable element. Elements of each type encode at least one polypeptide a transposase, required for their own transposition. Transposons often further include genes coding for function unrelated to transposition, for example, antibiotic resistance genes.

Transposons can be divided into two classes according to their structure. First, compound or composite transposons have copies of an insertion sequence element at each end, usually in an inverted orientation. These transposons require transposases encoded by one of their terminal IS elements. The second class of transposon have terminal repeats of about 30 base pairs and do not contain sequences from IS elements.

Transposition usually is either conservative or replicative, although in some cases it can be both. In replicative transposition, one copy of the transposing element remains at the donor site, and another is inserted at the target site. In conservative transposition, the transposing element is excised from one site and inserted at another.

Eukaryotic elements also can be classified according to their structure and mechanism of transportation. The primary distinction is between elements that transpose via an RNA intermediate, and elements that transpose directly from DNA to DNA.

Elements that transpose via an RNA intermediate often are referred to as retrotransposons, and their most characteristic feature is that they encode polypeptides that are believed to have reverse transcriptionase activity. There are two types of retrotransposon. Some resemble the integrated proviral DNA of a retrovirus in that they have long direct repeat sequences, long terminal repeats (LTRs), at each end. The similarity between these retrotransposons and proviruses extends to their coding capacity. They contain sequences related to the gag and pol genes of a retrovirus, suggesting that they transpose by a mechanism related to a retroviral life cycle. Retrotransposons of the second type have no terminal repeats. They also code for gag- and pol-like polypeptides and transpose by reverse transcription of RNA intermediates, but do so by a mechanism that differs from that or retrovirus-like elements. Transposition by reverse transcription is a replicative process and does not require excision of an element from a donor site.

Transposable elements are an important source of spontaneous mutations, and have influenced the ways in which genes and genomes have evolved. They can inactivate genes by inserting within them, and can cause gross chromosomal rearrangements either directly, through the activity of their transposases, or indirectly, as a result of recombination between copies of an element scattered around the genome. Transposable elements that excise often do so imprecisely and may produce alleles coding for altered gene products if the number of bases added or deleted is a multiple of three.

Transposable elements themselves may evolve in unusual ways. If they were inherited like other DNA sequences, then copies of an element in one species would be more like copies in closely related species than copies in more distant species. This is not always the case, suggesting that transposable elements are occasionally transmitted horizontally from one species to another.

ii) Chemical Mutagenesis

Chemical mutagenesis offers certain advantages, such as the ability to find a full range of mutant alleles with degrees of phenotypic severity, and is facile and inexpensive to perform. The majority of chemical carcinogens produce mutations in DNA. Benzo[a]pyrene, N-acetoxy-2-acetyl aminofluorene and aflotoxin B1 cause GC to TA transversions in bacteria and mammalian cells. Benzo[a]pyrene also can produce base substitutions such as AT to TA. N-nitroso compounds produce GC to AT transitions. Alkylation of the O4 position of thymine induced by exposure to n-nitrosoureas results in TA to CG transitions.

A high correlation between mutagenicity and carcinogenity is the underlying assumption behind the Ames test (McCann et al., 1975) which speedily assays for mutants in a bacterial system, together with an added rat liver homogenate, which contains the microsomal cytochrome P450, to provide the metabolic activation of the mutagens where needed.

In vertebrates, several carcinogens have been found to produce mutation in the ras protooncogene. N-nitroso-N-methyl urea induces mammary, prostate and other carcinomas in rats with the majority of the tumors showing a G to A transition at the second position in codon 12 of the Ha-ras oncogene. Benzo[a]pyrene-induced skin tumors contain A to T transformation in the second codon of the Ha-ras gene.

iii) Radiation Mutagenesis

The integrity of biological molecules is degraded by the ionizing radiation. Adsorption of the incident energy leads to the formation of ions and free radicals, and breakage of some covalent bonds. Susceptibility to radiation damage appears quite variable between molecules, and between different crystalline forms of the same molecule. It depends on the total accumulated dose, and also on the dose rate (as once free radicals are present, the molecular damage they cause depends on their natural diffusion rate and thus upon real time). Damage is reduced and controlled by making the sample as cold as possible.

Ionizing radiation causes DNA damage and cell killing, generally proportional to the dose rate. Ionizing radiation has been postulated to induce multiple biological effects by direct interaction with DNA, or through the formation of free radical species leading to DNA damage (Hall, 1988). These effects include gene mutations, malignant transformation, and cell killing. Although ionizing radiation has been demonstrated to induce expression of certain DNA repair genes in some prokaryotic and lower eukaryotic cells, little is known about the effects of ionizing radiation on the regulation of mammalian gene expression (Borek, 1985). Several studies have described changes in the pattern of protein synthesis observed after irradiation of mammalian cells. For example, ionizing radiation treatment of human malignant melanoma cells is associated with induction of several unidentified proteins (Boothman et al., 1989). Synthesis of cyclin and co-regulated polypeptides is suppressed by ionizing radiation in rat REF52 cells, but not in oncogene-transformed REF52 cell lines (Lambert and Borek, 1988). Other studies have demonstrated that certain growth factors or cytokines may be involved in x-ray-induced DNA damage. In this regard, platelet-derived growth factor is released from endothelial cells after irradiation (Witte et al., 1989).

In the present invention, the term “ionizing radiation” means radiation comprising particles or photons that have sufficient energy or can produce sufficient energy via nuclear interactions to produce ionization (gain or loss of electrons). An exemplary and preferred ionizing radiation is an x-radiation. The amount of ionizing radiation needed in a given cell generally depends upon the nature of that cell. Typically, an effective expression-inducing dose is less than a dose of ionizing radiation that causes cell damage or death directly. Means for determining an effective amount of radiation are well known in the art.

In a certain embodiments, an effective expression inducing amount is from about 2 to about 30 Gray (Gy) administered at a rate of from about 0.5 to about 2 Gy/minute. Even more preferably, an effective expression inducing amount of ionizing radiation is from about 5 to about 15 Gy. In other embodiments, doses of 2-9 Gy are used in single doses. An effective dose of ionizing radiation may be from 10 to 100 Gy, with 15 to 75 Gy being preferred, and 20 to 50 Gy being more preferred.

Any suitable means for delivering radiation to a tissue may be employed in the present invention in addition to external means. For example, radiation may be delivered by first providing a radiolabeled antibody that immunoreacts with an antigen of the tumor, followed by delivering an effective amount of the radiolabeled antibody to the tumor. In addition, radioisotopes may be used to deliver ionizing radiation to a tissue or cell.

iv) In Vitro Scanning Mutagenesis

Random mutagenesis also may be introduced using error prone PCR (Cadwell and Joyce, 1992). The rate of mutagenesis may be increased by performing PCR in multiple tubes with dilutions of templates.

One particularly useful mutagenesis technique is alanine scanning mutagenesis in which a number of residues are substituted individually with the amino acid alanine so that the effects of losing side-chain interactions can be determined, while minimizing the risk of large-scale perturbations in protein conformation (Cunningham et al., 1989).

In recent years, techniques for estimating the equilibrium constant for ligand binding using minuscule amounts of protein have been developed (Blackburn et al., 1991; U.S. Pat. Nos. 5,221,605 and 5,238,808). The ability to perform functional assays with small amounts of material can be exploited to develop highly efficient, in vitro methodologies for the saturation mutagenesis of antibodies. The inventor bypassed cloning steps by combining PCR mutagenesis with coupled in vitro transcription/translation for the high throughput generation of protein mutants. Here, the PCR products are used directly as the template for the in vitro transcription/translation of the mutant single chain antibodies. Because of the high efficiency with which all 19 amino acid substitutions can be generated and analyzed in this way, it is now possible to perform saturation mutagenesis on numerous residues of interest, a process that can be described as in vitro scanning saturation mutagenesis (Burks et al., 1997).

In vitro scanning saturation mutagenesis provides a rapid method for obtaining a large amount of structure-function information including: (I) identification of residues that modulate ligand binding specificity, (ii) a better understanding of ligand binding based on the identification of those amino acids that retain activity and those that abolish activity at a given location, (iii) an evaluation of the overall plasticity of an active site or protein subdomain, (iv) identification of amino acid substitutions that result in increased binding.

v) Random Mutagenesis by Fragmentation and Reassembly

A method for generating libraries of displayed polypeptides is described in U.S. Pat. No. 5,380,721. The method comprises obtaining polynucleotide library members, pooling and fragmenting the polynucleotides, and reforming fragments therefrom, performing PCR amplification, thereby homologously recombining the fragments to form a shuffled pool of recombined polynucleotides.

C. Site-Directed Mutagenesis

Structure-guided site-specific mutagenesis represents a powerful tool for the dissection and engineering of protein-ligand interactions (Braisted and Wells, 1996), especially in the context of the present invention where specific mutations in cleavage sites are sought. The technique provides for the preparation of sequence variants by introducing one or more discrete nucleotide sequence changes into a selected nucleic acid.

Site-specific mutagenesis uses specific oligonucleotide sequences which encode the DNA sequence of the desired mutation, as well as a sufficient number of adjacent, unmodified nucleotides. In this way, a primer sequence is provided with sufficient size and complexity to form a stable duplex on both sides of the deletion junction being traversed. A primer of about 17 to 25 nucleotides in length is preferred, with about 5 to 10 residues on both sides of the junction of the sequence being altered.

The technique typically employs a bacteriophage vector that exists in both a single-stranded and double-stranded form. Vectors useful in site-directed mutagenesis include vectors such as the M13 phage. These phage vectors are commercially available and their use is generally well known to those skilled in the art. Double-stranded plasmids are also routinely employed in site-directed mutagenesis, which eliminates the step of transferring the gene of interest from a phage to a plasmid.

In general, one first obtains a single-stranded vector, or melts two strands of a double-stranded vector, which includes within its sequence a DNA sequence encoding the desired protein or genetic element. An oligonucleotide primer bearing the desired mutated sequence, synthetically prepared, is then annealed with the single-stranded DNA preparation, taking into account the degree of mismatch when selecting hybridization conditions. The hybridized product is subjected to DNA polymerizing enzymes such as E. coli polymerase I (Klenow fragment) in order to complete the synthesis of the mutation-bearing strand. Thus, a heteroduplex is formed, wherein one strand encodes the original non-mutated sequence, and the second strand bears the desired mutation. This heteroduplex vector is then used to transform appropriate host cells, such as E. coli cells, and clones are selected that include-recombinant vectors bearing the mutated sequence arrangement.

Comprehensive information on the functional significance and information content of a given residue of protein can best be obtained by saturation mutagenesis in which all 19 amino acid substitutions are examined. The shortcoming of this approach is that the logistics of multiresidue saturation mutagenesis are daunting (Warren et al., 1996, Zeng et al., 1996; Barbas et al., 1994; Yelton et al., 1995; Wong et al., 1996; Hilton et al., 1996). Hundreds, and possibly even thousands, of site specific mutants must be studied. However, improved techniques make production and rapid screening of mutants much more straightforward. See also, U.S. Pat. Nos. 5,798,208 and 5,830,650, for a description of “walk-through” mutagenesis.

Other methods of site-directed mutagenesis are disclosed in U.S. Pat. Nos. 5,220,007; 5,284,760; 5,354,670; 5,366,878; 5,389,514; 5,635,377; and 5,789,166.

D. Virus Transformation and Propagation

Targeted recombination has become a powerful tool to introduce mutations into the genome and determine their effects on protein function, virus replication and virus pathogenesis (Koetzner et al., 1992; Masters et al., 1994; Fischer et al., 1997; Lavi et al., 1998; Leparc-Goffart et al., 1998; Phillips et al., 1999; Sanchez et al., 1999; Phillips et al., 2001; de haan et al., 2002; Sarma et al., 2002). However, the available recombination constructs and methodologies have thus far limited the use of targeted recombination, and have not been employed to examine mutations in the replicase gene.

The inventor has previously collaborated in the development of a system for assembly of full-length MHV genome cDNA, generation of genome length RNA, and recovery of virus from transfected cells (Schaad et al., 1990; Yount et al., 2002). In this process, seven contiguous cDNA clones that spanned the 31.5-kb genome of mouse hepatitis virus strain A59 (MHV-A59) were isolated. The ends of the cDNAs were engineered with unique junctions and assembled with only the adjacent cDNA subclones, resulting in an intact MHV-A59 cDNA construct of about 31.5 kb in length. The interconnecting restriction site junctions that are located at the ends of each cDNA are systematically removed during the assembly of the complete full-length cDNA product, allowing reassembly without the introduction of nucleotide changes.

RNA transcripts derived from the full-length MHV-A59 construct were infectious, although transfection frequencies were enhanced 10- to 15-fold in the presence of transcripts encoding the nucleocapsid protein N. Plaque-purified virus derived from the infectious construct replicated efficiently and displayed similar growth kinetics, plaque morphology, and cytopathology in murine cells as did wild-type MHV-A59. Molecularly cloned viruses recognized the MHV receptor (MHVR) for docking and entry, and pretreatment of cells with monoclonal antibodies against MHVR blocked virus entry and replication. Cells infected with molecularly cloned MHV-A59 virus expressed replicase (gene 1) proteins identical to those of laboratory MHV-A59. Importantly, the molecularly cloned viruses contained three marker mutations that had been derived from the engineered component clones.

Using this process, full-length infectious constructs of MHV-A59 and other coronaviruses with genetic modifications of may be created. In fact, the method has the potential to be used to construct viral, microbial, or eukaryotic genomes approaching several million base pairs in length and used to insert restriction sites at any given nucleotide in a microbial genome. A similar system approach was used previously with TGEV, including the insertion of heterologous genes into the TGEV genome (Yount, 2000; Curtis et al., 2002). The inventor described herein the use of this same assembly approach to introduce five different mutations into the MHV p28/p65 cleavage site (CS1). While the approaches are similar, it was not usually necessary with MHV to introduce mutations and new restriction sites into the wild-type virus genome to direct the assembly cascade. Rather, type IIS restriction endonuclease Esp3I or BsmB1 sites can be used to create the unique interconnecting junctions, and yet be subsequently removed from the final assembly product, allowing for the reconstruction of an intact wild-type sequence. This approach avoids the introduction of nucleotide changes that are normally associated with building a full-length cDNA product of a viral genome.

The use of non-palindromic restriction sites also provides other novel recombinant DNA applications. For example, by PCR, it is be possible to insert Esp3I or a related non-palindromic restriction site at any given nucleotide in a viral genome and use the variable domain for simple and rapid site-specific mutagenesis. By orienting the restriction sites as “No See'm”, the sites are removed during reassembly, leaving only the desired mutation in the final DNA product. The dual properties of strand specificity and a variable end overhang that can be tailored to match any sequence allow for Esp3I sites to be engineered as universal connectors that can be joined with any other 4-nucleotide restriction site overhang (e.g., EcoRI, PstXI and BamHI). Alternatively, No See'm sites can be used to insert foreign genes into viral, eukaryotic, or microbial genomes or vectors, simultaneously removing all evidence of the restriction sites that were used in the recombinant DNA manipulation.

In order to remove preexisting Esp3I sites that resided within the MHV-A59 genome sequence, silent mutations were created. This helped to distinguish between molecularly cloned and wild-type viruses. In one instance, the Esp3I site at position 4875 was removed because it left a TTAA overhang that would have prevented the directionality of assembly. The other Esp3I sites were removed to minimize the total number of MHV-A59 subclones used in the assembly cascade. In two instances, silent mutations were inserted into the Esp3I overhang to maximize sequence specificity and directionality at a particular junction, but this could be circumvented by choosing slightly different junction sites. Clearly, each virus sequence will need to be evaluated for the need for similar changes.

cDNA cassettes can be ligated systematically as previously described for TGEV, or simultaneously as described herein. Although numerous incomplete assembly intermediates occur were evident, the inventor has found that simultaneous ligation of seven cDNAs will result in full-length cDNA, thereby simplifying the complexity of the assembly strategy. There is no evidence to indicate that this approach might introduce spurious mutations or genome rearrangements from aberrant assembly cascades. And while it is possible that such variants might arise following RNA transfection (as a consequence of high-frequency MHV RNA recombination between incomplete and genome-length transcripts), it is highly likely that such variants would be replication impaired and rapidly outcompeted by wild-type virus. A second limitation is that the yield of full-length cDNA product is reduced, resulting in less robust transfection efficiencies than those of the more traditional systematic assembly method. This downside is more than compensated by the reduced complexity in many cases.

Using this same approach, the inventor collaborated in the establishment of a reverse genetic system for SARS-CoV (Yount, et al., 2003).

### V. Vaccines

A. Formulations and Administration

The present invention provides for Nidovirus vaccine formulations. Such compositions will generally be dissolved or dispersed in a pharmaceutically acceptable carrier or aqueous medium. There are numerous examples of vaccine formulations in the literature, and one of skill in the art will be capable of formulating such vaccines.

The phrases “pharmaceutically acceptable” or “pharmacologically acceptable” refer to molecular entities and compositions that do not produce an adverse, allergic, or other untoward reaction when administered to an animal, or human, as appropriate. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art.

The vaccines of the present invention can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and, the preparations can also be emulsified. The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques. In certain cases, the therapeutic formulations of the invention also may be prepared in forms suitable for oral or intranasal administration.

An effective amount of the vaccine is determined based on the intended goal. The term “unit dose” or “dosage” refers to physically discrete units suitable for use in a subject, each unit containing a predetermined-quantity of the composition calculated to produce the desired responses, discussed above, in association with its administration, i.e., the appropriate route and treatment regimen. Precise amounts of the vaccine composition also depend on the judgment of the practitioner and are peculiar to each individual. Factors affecting dose include physical and clinical state of the patient, the route of administration, the intended goal of treatment (alleviation of symptoms versus cure) and the potency, stability, and toxicity of the particular substance.

The following is a listing of references describing various live vaccines, the relevant contents of which (formulations and administration) are hereby incorporated by reference:
U.S. Pat. Nos. 6,479,056; 6,444,445; 6,306,400; 6,296,854; 6,231,871; 6,217,882; 6,159,477; 6,153,199; 6,136,325; 6,077,516; 6,051,237; 6,045,803; 6,039,958; 6,039,941; 6,033,670; 5,993,822; 5,980,906; 5,958,423; 5,948,411; 5,871,742; 5,869,036; 5,792,452; 5,733,555; 5,733,554; 5,651,972; 5,632,989; 5,626,850; 5,580,557; 5,436,001; 5,310,668; 5,149,531; 5,068,104; 5,037,650; 5,024,836; 5,006,335; 4,985,244; 4,980,162; 4,808,404; 4,770,875; 4,762,711; 4,752,474; 4,673,572; 4,645,665; 4,624,850; 4,590,072; 4,555,401; 4,554,158; 4,472,378; 4,456,588; 4,324,861; 4,311,797; 4,235,876; 4,004,974

B. Additional Agents

In addition to the inactive agents discussed above, the vaccine may comprise, or may be given in conjunction with, a supplemental agent. One example is an immunostimulant.

### VI. Examples

The following examples are included to further illustrate various aspects of the invention. It should be appreciated by those of skill in the art that the techniques disclosed in the examples which follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Polyprotein Structure

Amino terminal proteins and papain-like proteinases. For group 2 coronaviruses, such as MHV, and for SARS-CoV, the amino-terminal third of the complete 800 kDa replicase polyprotein contains the nsp1, nsp2, and nsp3 domains (FIG. 2). This portion of the replicase is most variable for all coronaviruses. For MHV and SARS-CoV, the nsp1, 2, and 3 proteins are cleaved by two (MHV) or one (SARS-CoV) PLP(s) at cleavage sites (CS) that are relatively conserved. The cleavage site specificities for PLP1 and PLP2 of MHV have been well characterized in vitro (Dong and Baker, 1994; Hughes et al., 1995; Bonilla, et al., 1995) Based on those studies, the inventor used reverse genetic approaches to show that for MHV, alteration or deletion of the first two cleavage sites (CS1 and CS2) allows recovery of viable mutants with variable degrees of impairment of replication, and that may contribute to virulence attenuation in mice (Denison et al., 2004) The PLP enzymes responsible for cleavage of MHV CS1, CS2, and CS3 (at the end of nsp3) are located in nsp3. The SARS-CoV PLP is most similar in location, sequence and probable cleavage site specificity to the PLP2 of MHV and other group 2 coronaviruses (Snijder et al., 2003). For MHV, the inventor has shown that abolition of PLP1 activity allows recovery of viable viruses with CS1 and CS2 cleavage or lack thereof. Other than PLP activity, nothing is known of the functions or requirements for nsp1, nsp2, and nsp3 in replication.

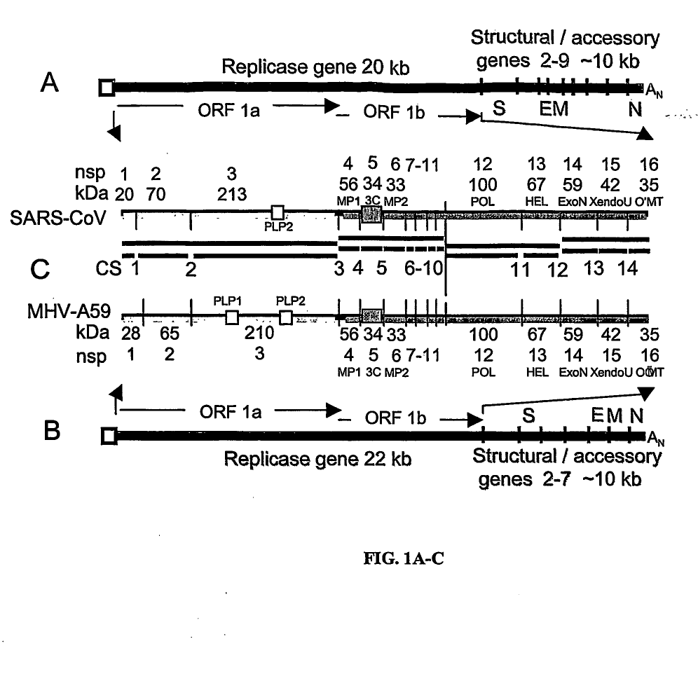

Nsp4-13: Processing and Function. For all coronaviruses, the protein regions from nsp4 to nsp16 are completely conserved in their organization, numbers, and relative sizes and are up to 80% identical or similar in amino acid sequence by direct comparison and are likely all cleaved by 3CLpro. In addition, the known, probable, and predicted functions are conserved across coronaviruses as well (FIGS. 1A-B). Nsp4 and 6 are known integral membrane proteins that likely participate in replication complex formation and 3CLpro function (nsp5). Nsp5 is responsible for all cleavages of nsp5-16. The crystal structures of the nsp5-3CLpro of human coronavirus 229E (HCoV-229E) and SARS-CoV have recently been reported (Anand et al., 2003; Yang et al., 2003). Inhibition of 3CLpro activity blocks processing of nsps5-16 and rapidly aborts viral RNA synthesis and growth of infectious virus (Kim et al., 1995). The nsp7-10 proteins are four small proteins that comprise the carboxy-terminus of ORF1a, localize to replication complexes in the cytoplasm, and have 60 to 80% conservation of sequence and cleavage sites across the coronaviruses (Bost et al., 2000; Snijder et al., 2003). Studies in the inventor's laboratory have shown specific interactions of MHV nsp7 and nsp10 with nsp1, suggesting roles as complexes (not shown). Nsp 11 represents the predicted 18 amino acids polypeptide that would result from failure of the ribosomal frameshift into ORF1b. There is no experimental evidence for this protein in infected cells. The nsp12 protein is the putative RNA-dependent RNA polymerase (pol) that spans the ribosomal frameshift. The expression and determinants of targeting of MHV pol to replication complexes was demonstrated by the inventor's laboratory (Brockway et al., 2003). However, biochemical or genetic evidence for pol function has not been reported. Nsp13 is a protein with demonstrated ATPase and RNA helicase (hel) activities for HCoV-229E and SARS-CoV and likely participates in RNA synthesis (Heusipp et al., 1997)

### Example 2

### Engineering of Mutants

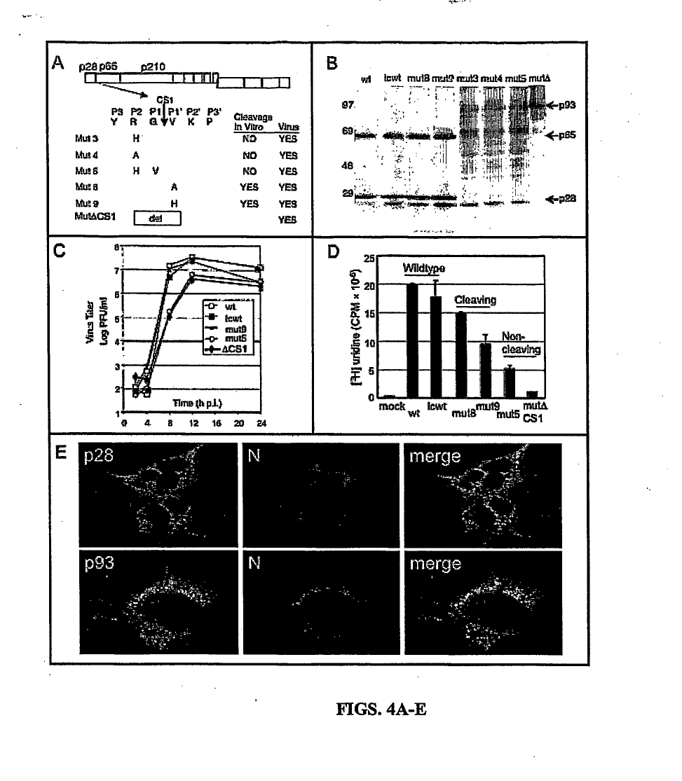

The inventor has used the reverse genetic system for MHV (FIG. 3) to introduce mutations predicted to abolish or retain cleavage at the replicase cleavage site 1 (CS1) between p28 (nsp1) and p65 (nsp2), as well as to create a three amino-acid deletion at P2-P1′ (ΔRGV) (FIG. 4A). Of the viruses recovered, viruses with predicted inhibition of CS1 cleavage (mut3, 4, 5, and ΔCS1) had small plaques compared to wild-type viruses (not shown). When viruses were assessed for replicase polyprotein processing, mutants predicted to retain cleavage (mut8, 9) demonstrated complete cleavage at CS1, resulting in detectable p28 and p65 proteins (FIG. 4B). In contrast, mutants predicted to abolish cleavage had no detectable p28 or p65, while a new 93 kDa protein was detected, consistent with an uncleaved p28-p65 precursor. Analysis of virus growth demonstrated that cleavage-competent mutants grew with wild-type kinetics and peak titer, while non-cleaving mutants, including ΔCS1, had delayed kinetics of growth and were consistently 1 to 1.5 log reduced in peak titer (FIG. 4C). When RNA synthesis was analyzed, the CS1 noncleaving mutants had significant defects in total viral RNA synthesis up to 90% (FIG. 4D), suggesting that the defect in virus growth may be due to an inhibition of RNA synthesis, and that the p28 and/or p65 proteins may have direct functions in RNA synthesis that require full cleavage for activity. The inventor subsequently demonstrated by Northern blot that the impairment of RNA synthesis is global, affecting all RNA species equally (Denison et al., 2004). When proteins were probed by IFA, the uncleaved p93 demonstrated less complete colocalization with N protein and replication complexes than the cleaved p28 and p65 proteins (FIG. 4E).

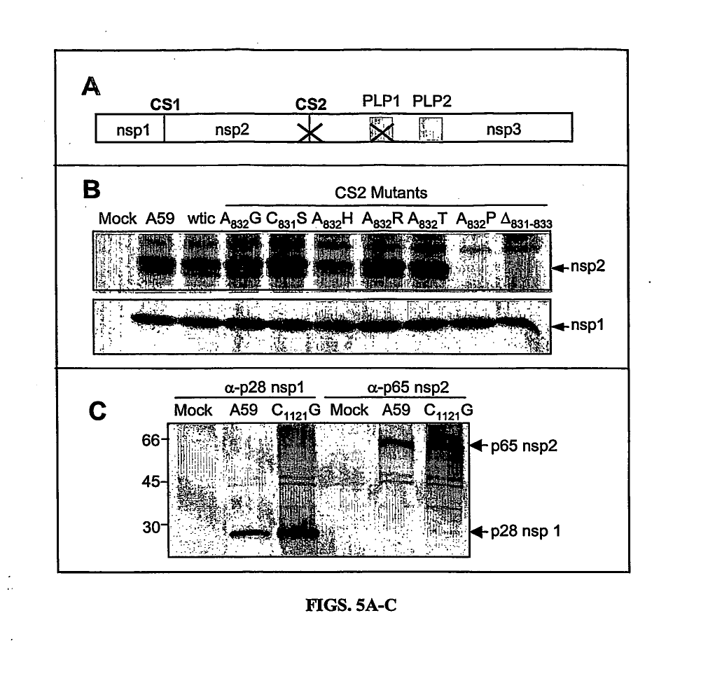

Mutations were introduced in MHV CS2 (P2₈₃₁Cys, P1₈₃₂A1a, and P1′₈₃₃Gly) designed to retain or abolish cleavage based on in vitro studies (FIGS. 5A-B) (Dong and Baker, 1994; Hughes et al., 1995). In vitro, while most mutations at P2₈₃₁Cys allowed cleavage to occur, P1₈₃₂Ala of CS2 could only be substituted with small, uncharged residues and still retain cleavage. In agreement with this, a Cys₈₃₁Ser change and a Ala₈₃₂Gly substitution allowed recovery of viable virus with wild-type cleavage of nsp2 (p65). Substitution of Ala₈₃₂ by Pro, or deletion of P2Cys, P1A1a and P1′Gly also allowed recovery of viable virus, but with completely abolished nsp2 cleavage. Surprisingly, when other bulky, charged, or non-conservative residues were substituted for P1A1a (His, Arg, Thr) viable virus was recovered that had wild-type or near wild-type cleavage of nsp2 at CS2. Based on these findings, the inventor concludes that while MHV likely cleaves between residues 832 and 833 in the context of infection, the residues it requires for cleavage site specificity differ substantially from those identified in vitro. This difference may be a result of polyprotein folding differences or PLP2 dominance over PLP1 in CS2 processing in the context of infection. These results demonstrate both the feasibility and importance of studying the cleavage and determinants of cleavage in the context of the viral genome and infection.

When the catalytic Cys₁₁₂₁ of PLP1 was substituted with Gly, low-level CPE was observed but no virus was recovered until seven days post-electroporation, at which time productive syncytia appeared and viable virus was recovered (FIG. 5C). The recovered replication revertant population was sequenced and found to contain the mutation Cys₁₁₂₁Gly mutation and no other changes in the 1 kb PLP1 domain. When cells infected with the mutant were immunoprecipitated for nsp1-p28 and nsp2-p65, both proteins were processed. This result demonstrates the feasibility of recovery and analysis of highly detrimental mutations as revertants, and also suggests that the MHV mutant that may be “SARS-CoV”—like by cleaving CS1, CS2, and CS3 with a single functional PLP activity.

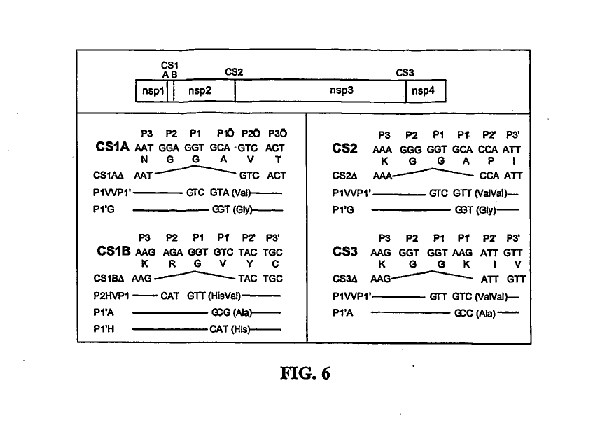

The inventor has successfully introduced mutations and deletions in the SARS-CoV genome at putative CS1A, a possible alternative CS1B, CS2, and CS3 in the cloned fragment A, and will use these for assembly of virus mutants (FIG. 6).

### Example 3

### Future Studies

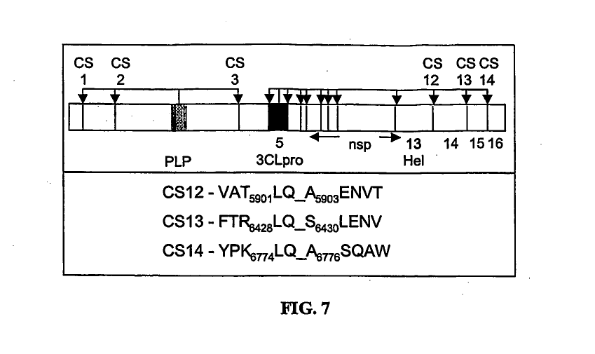

In SARS-CoV, mutations and deletions will be engineered in cleavage sites flanking nsp1, nsp2, and nsp3 (CS1, CS2, and CS3) that are predicted to be cleaved by the PLP. Mutations predicted to allow cleavage will be compared with mutations and deletions predicted to abolish cleavage for the ability to recover viable viruses. Specific sites processed by 3CLpro (CS12, CS13, and CS14) will be mutated individually or as groups to recapitulate the uncleaved intermediate precursors detected during viral infection (FIG. 7). Viable mutants will be analyzed for growth, plaque size and cytopathic effect, protein processing, and RNA synthesis as outlined above. Mutants with wild-type phenotypes will be passaged to determine the stability of introduced changes. Mutants impaired in replication will be passaged with selection for reversion, and phenotypic revertants will be sequenced to identify compensating mutations.

The CS1, CS2, and CS3 cleavage sites will be targeted for mutagenesis and deletion. Studies with the MHV model suggest that during normal virus infection there may be intermediate precursors of nsp1, 2, and 3 that may support virus functions to some extent. In addition, deletion of MHV CS1, CS2, or both, allows recovery of viable mutants with different degrees of impairment in virus growth and RNA synthesis, and clear evidence for viability with cleavage site deletions of CS1 and CS2. Importantly, several CS1 and CS2 mutants that allow cleavage of the sites have wild-type virus growth but subtle impairment of viral RNA synthesis, suggesting independent effects of the mutations on protein function independent of the effect on cleavage site processing. Thus, the inventor anticipates that these experiments will define processing events and residues that affect viral growth and RNA synthesis to different extents in culture.

In addition to establishing viable mutants for testing in replication, the experiments will confirm the functional cleavage sites and define residues important for cleavage and those that may serve other functions at the termini of the proteins. However, the SARS-CoV PLP has already been demonstrated in vitro to cleave a peptide containing CS2. In addition, while CS1 has been predicted to be ₁₈₀G_A₁₈₁, the polyprotein also retains an RG_V tripeptide of the identical sequence and similar location to the MHV RG_V CS1. While preliminary studies identify an nsp1 protein of 20 kDa, consistent with cleavage at ₁₈₀G_A₁₈₁, it is possible that more than one site may be used by for cleavage, or may function as alternative cleavage sites.

Mutations and deletions of cleavage sites have already been cloned and confirmed in genome fragments as shown in FIG. 7. Two different sites for possible CS1 have been mutated: ₁₇₉GG_A₁₈₁ (CS1A) and ₂₂₆RG_V228. For CS2, the predicted ₈₁₇GG_A₈₁₉ has been mutated and deleted. The changes and deletions that have been cloned in Fragment A are based on predicted specificity and our results with changes that retain or abolish cleavage in the context of MHV infection, and the fact that deletions of P2,P1_P1′ are tolerated in recovered viable mutants. In addition, to maximize resistance to same site reversion, all changes will be at least two nucleotides and for the most part multiple nucleotides in at least two codons.

Full-length mutated genome will be used to electroporate cells, followed by recovery and analysis of viable viruses. Based on the MHV results, the inventor anticipates recovery of viable mutants with either retention or abolition of cleavage at CS1 or CS2. Further, determining whether PLP cleaves at CS1A, CS1B, or both will provide insights into the relationship with other coronaviruses and the evolution of proteinase-cleavage site interactions.

There are 10 possible 3CLpro cleavage sites in the replicase polyprotein. With the exception of the putative RNA-dependent RNA polymerase (nsp12) and helicase (nsp13), the functions are either predicted or not known. However, the high degree of conservation of the sites across SARS-CoV and coronaviruses of every known group indicates stably evolved roles in regulation of protein availability and possibly function. Protein processing studies from MHV and IBV suggest several possible intermediate precursors to the final mature replicase proteins (see FIGS. 1A-C). These can be grouped as nsp4-10, nsp12-13, and nsp14-16.

The inventor will investigate the processing and cleavage sites of nsp14-16 (CS12, CS13, and CS14) in SARS-CoV replication based on the following criteria: (1) the proteins have putative functions as RNA processing enzymes; (2) they do not represent putative core enzymatic functions that would be predicted to be easily altered in a precursor form; (3) studies of other coronaviruses suggest that nsp14-16 exist as a detectable intermediate precursor in infected cells; and (4) recovery of viable mutants will provide a foundation for construction of viruses with independent mutations across the replicase.

As shown in FIG. 7, the P2, P1, and P1′ residues that constitute the cleavage sites (LQ_(A,S) are almost completely conserved. Characterized 3CLpro cleavage sites appear to have flexibility at the P1′ position (Ala, Val, Ser, Gly, Asn). However, it is not known if the differences in P1′ residues are critical for processing in their local contexts or may regulate the order of cleavages in some fashion. In addition, it is possible that the P1′ residues may serve roles in the protein functions independent of their roles in cleavage site processing.

The inventor will delete P2-P1′ and make substitutions at each residue individually and in combination that would have probability of maintaining or abolishing cleavage. All mutations of P1-Glu are expected to abolish cleavage at any site, as will deletions of P2-P1′. Further, it is hypothesized that mutations that retain cleavage will be viable, and that cleavage knockout mutants will be viable but with varying degrees of replication impairment. If deletions or mutations predicted to block cleavage are lethal for growth, changes will be introduced that have been shown to allow cleavage at other 3CLpro cleavage sites, such as P2-Leu to Ile or Val or P1′ Ala/Ser to Ser/Ala, Gly, or Cys. This will determine both if there is limited allowed variation at the sites for cleavage and if there is impact on replication independent of the effect on protein processing. The inventor will also establish multiple passage of electroporated cells to select for revertants with wild-type replication. Any mutations or deletions that allow recovery of stable mutants with wild-type growth will be combined with similar mutants in CS1, CS2, or CS3 to determine if alteration of multiple cleavage sites for PLP and 3CLpro can still retain growth in culture. Further, reagents and clones are becoming available to allow one to rapidly address other 3CLpro cleavage sites, specifically, the region of four small proteins at the end of ORF1a (nsp7-10).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the scope of the invention as defined by the appended claims.

### VII. References

The following references, to the extent that they provide exemplary procedural or other details supplementary to those set forth herein, are specifically incorporated herein by reference:
U.S. Pat. No. 4,004,974
U.S. Pat. No. 4,235,876
U.S. Pat. No. 4,311,797
U.S. Pat. No. 4,324,861
U.S. Pat. No. 4,456,588
U.S. Pat. No. 4,472,378
U.S. Pat. No. 4,554,158
U.S. Pat. No. 4,555,401
U.S. Pat. No. 4,590,072
U.S. Pat. No. 4,624,850
U.S. Pat. No. 4,645,665
U.S. Pat. No. 4,673,572
U.S. Pat. No. 4,752,474
U.S. Pat. No. 4,762,711
U.S. Pat. No. 4,770,875
U.S. Pat. No. 4,808,404
U.S. Pat. No. 4,980,162
U.S. Pat. No. 4,985,244
U.S. Pat. No. 5,006,335
U.S. Pat. No. 5,024,836
U.S. Pat. No. 5,037,650
U.S. Pat. No. 5,068,104
U.S. Pat. No. 5,149,531
U.S. Pat. No. 5,220,007
U.S. Pat. No. 5,221,605
U.S. Pat. No. 5,238,808
U.S. Pat. No. 5,284,760
U.S. Pat. No. 5,310,668
U.S. Pat. No. 5,354,670
U.S. Pat. No. 5,366,878
U.S. Pat. No. 5,380,721
U.S. Pat. No. 5,389,514
U.S. Pat. No. 5,436,001
U.S. Pat. No. 5,580,557
U.S. Pat. No. 5,626,850
U.S. Pat. No. 5,632,989
U.S. Pat. No. 5,635,377
U.S. Pat. No. 5,651,972
U.S. Pat. No. 5,733,554
U.S. Pat. No. 5,733,555
U.S. Pat. No. 5,789,166
U.S. Pat. No. 5,792,452
U.S. Pat. No. 5,798,208
U.S. Pat. No. 5,830,650
U.S. Pat. No. 5,869,036
U.S. Pat. No. 5,871,742
U.S. Pat. No. 5,948,411
U.S. Pat. No. 5,958,423
U.S. Pat. No. 5,980,906
U.S. Pat. No. 5,993,822
U.S. Pat. No. 6,033,670
U.S. Pat. No. 6,039,941
U.S. Pat. No. 6,039,958
U.S. Pat. No. 6,045,803
U.S. Pat. No. 6,051,237
U.S. Pat. No. 6,077,516
U.S. Pat. No. 6,136,325
U.S. Pat. No. 6,153,199
U.S. Pat. No. 6,159,477
U.S. Pat. No. 6,217,882
U.S. Pat. No. 6,231,871
U.S. Pat. No. 6,296,854
U.S. Pat. No. 6,306,400
U.S. Pat. No. 6,444,445
U.S. Pat. No. 6,479,056
Anand et al., Science, 300:1763-1767, 2003
Baker et al, J. Virol., 67:6056-6063, 1993.
Barbas et al., Proc. Natl. Acad. Sci. USA, 91(9):3809-3813, 1994.
Blackburn et al., J. Lipid. Res., 32(12):1911-1918, 1991.
Bonilla et al., J. Virol., 71:900-909, 1997.
Bonilla et al., Virology, 209:489-497, 1995.
Boothman et al., Cancer Res., 49(11):2871-2878, 1989.
Borek, Carcinog. Compr. Surv., 10:303-316, 1985.
Bost et al., J. Virol. 74:3379-3387, 2000.
Brockway et al., J. Virol. 77:10515-10527.
Boursnell et al., J. Gen. Virol., 68:57-77, 1987.
Braisted and Wells, Proc. Natl. Acad. Sci. USA, 93(12):5688-5692, 1996.
Burks et al., Proc. Natl. Acad. Sci. USA, 94(2):412-417, 1997.
Caldwell and Joyce, PCR Methods Appl., 2(1):28-33, 1992.
Chouljenko et al., J. Gen. Virol., 82:2927-2933, 2001.
Cooley et al., Science, 239(4844):1121-1128, 1988.
Cunningham and Wells, Science, 244(4908):1081-1085, 1989.
Curtis et al., J. Virol., 76:1422-1434, 2002.
de Haan et al., Virology, 296:177-189, 2002.
Denison et al., J. Virol. In press 2004.
Dong and Baker, Virology, 204:541-549, 1994.
Eleouet et al., Virology, 206:817-822, 1995.
Fischer et al., J. Virol., 71:5148-5146, 1997.
Hall, Genetics, 120(4):887-897, 1988.
Herold et al., J. Biol. Chem., 274:14918-14925, 1999.
Herold et al., Virology, 195:680-691, 1993.
Heusipp et al., J. Virol., 71:3992-3997.
Hilton et al., J. Biol. Chem., 271(9):4699-4708, 1996.
Hughes et al., J. Virol., 69:809-813, 1995.
Kanjanahaluethai and Baker, J. Virol., 74:7911-7921, 2000.
Kanjanahaluethai et al., Adv. Exp. Med. Biol., 494:267-273, 2001.
Kim et al., Virology, 208:1-8, 1995
Koetzner et al., J. Virol., 66:1841-1848, 1992.
Koncz et al., EMBO J., 9(5):1337-1346, 1990.
Lambert and Borek, J. Natl. Cancer Inst., 80(18):1492-1497, 1988.
Lavi et al., Adv. Exp. Med. Biol., 440:543-547, 1998.
Lee et al., Virology, 180:567-582, 1991.
Leparc-Goffart et al., J. Virol., 72:9628-9636, 1998.
Marks et al., Symp. Soc. Exp. Biol., 45:77-87, 1991.
Masters et al., J. Virol., 68:328-337, 1994.
McCann et al., Proc. Natl. Acad. Sci. USA, 72(3):979-983, 1975.
Oppenheimer et al., Cell, 67(3):483-493, 1991.
Phillips et al., J. Neurovirol., 7:421-431, 2001.
Phillips et al., J. Virol., 73:7752-7760, 1999.
Sanchez et al., J. Virol., 73:7607-7618, 1999.
Sarma et al., J Neurovirol., 8:381-391, 2002.
Schaad et al., Virology, 177:634-645, 1990.
Schiller et al., Virology, 242:288-302, 1998.
Schmidt et al., Science, 238(4829):960-963, 1987.
Snijder et al., J Mol Biol 331:991-1004, 2003
Sommer et al. EMBO J., 9(3):605-613, 1990.
Tijms et al., Proc. Natl. Acad. Sci. USA, 98:1889-1894, 2001.
Warren et al., Biochemistry, 35(27):8855-8862, 1996.
Wells et al., J. Leukoc. Biol., 59(1):53-60, 1996.
Witte et al., Cancer Res., 49(18):5066-5072, 1989.
Wong et al., J. Bacteriol., 178(8):2334-2342, 1996.
Yang et al., Proc. Natl. Acad. Sci. USA, 100:13190-13195
Yelton et al., J. Immunol., 155(4):1994-2004, 1995.
Yoo and Pei, Adv. Exp. Med. Biol., 494:73-76, 2001.
Yount et al., J. Virol., 76:11065-11078, 2002.
Yount, et al., J. Virol., 74:10600-10611, 2000.
Yount, et al., Proc. Natl. Acad. Sci. USA, 100:12995-13000, 2003.
Zeng et al., Biochemistry, 35(40):13157-13164, 1996.
Ziebuhr et al., J, Virol., 73(1):177-185, 1999.
Ziebuhr et al., J. Biol. Chem., 276:33220-33232, 2001.

1. A live, attenuated virus of the order Nidovirales, said virus characterized as comprising a genome encoding a replicase polyprotein comprising at least one proteinase cleavage site that exhibits reduced or no cleavage.

2. The virus of claim 1, wherein the virus is of the family Coronaviridae.

3. The virus of claim 2, wherein the virus is a coronavirus or a torovirus.

4. The virus of claim 3, wherein the coronavirus is avian infectious bronchitis virus, bovine coronavirus, canine coronavirus, feline infectious peritonitis virus, human coronavirus 229E, human coronavirus OC43, murine hepatitis virus, porcine epidemic diarrhea virus, porcine hemagglutinating encephalomyelitis virus, porcine transmissible gastroenteritis virus, rat coronavirus, turkey coronavirus, severe acute respiratory syndrome virus, or rabbit coronavirus.

5. The virus of claim 3, wherein the torovirus is Berne virus or Breda virus.

6. The virus of claim 1, wherein the virus is of the family Ateriviridae.

7. The virus of claim 6, wherein the virus is an aterivirus.

8. The virus of claim 7, wherein the aterivirus is equine arteritis virus, Lelystad virus or simian hemorrhagic fever virus.

9. The virus of claim 1, wherein said replicase polyprotein comprises at least a second proteinase cleavage site that exhibits reduced as compared to wild-type, or no cleavage.

10. The virus of claim 4, wherein the cleavage site is a C1-C14 cleavage site.

11. The virus of claim 4, wherein the cleavage site is a murine hepatitis virus p28-p65 or p65-p210 cleavage site.

12. The virus of claim 1, wherein the cleavage site exhibits reduced cleavage as compared to wild-type.

13. The virus of claim 1, wherein the cleavage site exhibits no cleavage.

14. The virus of claim 1, wherein the cleavage site contains an amino acid deletion, an amino acid insertion or an amino acid substitution.

15. The virus of claim 1, wherein the cleavage site is wild-type, but cleavage is reduced or eliminated by an allosteric mutation.

16. A method of inducing an anti-viral immune response in a host comprising administering to said host a live, attentuated vaccine of the order Nidovirales, said vaccine characterized as comprising a genome encoding a replicase polyprotein comprising at least one proteinase cleavage site that exhibits reduced or no cleavage.

17. The method of claim 16, wherein the vaccine is of the family Coronaviridae.

18. The method of claim 17, wherein the vaccine is a coronavirus or a torovirus.

19. The method of claim 18, wherein the coronavirus is avian infectious bronchitis virus, bovine coronavirus, canine coronavirus, feline infectious peritonitis virus, human coronavirus 229E, human coronavirus OC43, murine hepatitis virus, porcine epidemic diarrhea virus, porcine hemagglutinating encephalomyelitis virus, porcine transmissible gastroenteritis virus, rat coronavirus, turkey coronavirus, severe acute respiratory syndrome virus, or rabbit coronavirus.

20. The method of claim 18, wherein the torovirus is Berne virus or Breda virus.

21. The method of claim 16, wherein the vaccine is of the family Ateriviridae.

22. The method of claim 21, wherein the vaccine is an aterivirus.

23. The method of claim 22, wherein the aterivirus is equine arteritis virus, Lelystad virus or simian hemorrhagic fever virus.

24. The method of claim 16, wherein said replicase polyprotein comprises at least a second proteinase cleavage site that exhibits reduced as compared to wild-type, or no cleavage.

25. The method of claim 19, wherein the cleavage site is a C1-C14 cleavage site.

26. The method of claim 19, wherein the cleavage site is a murine hepatitis virus p28-p65 or p65-p210 cleavage site.

27. The method of claim 16, wherein the cleavage site exhibits reduced cleavage as compared to wild-type.

28. The method of claim 16, wherein the cleavage site exhibits no cleavage.

29. The method of claim 16, wherein the cleavage site contains an amino acid deletion, an amino acid insertion or an amino acid substitution.

30. The method of claim 16, wherein the cleavage site is wild-type, but cleavage is reduced or eliminated by an allosteric mutation.

31. The method of claim 16, wherein said vaccine is administered intravenously or subcutaneously.

32. The method of claim 16, further comprising immunostimulant.

33. The method of claim 16, wherein said host is a dog, a cow, a pig, a cat, a mouse, a rat, a horse, a chicken, a turkey, a monkey or a human.

34. A nidovirus genome, said genome encoding a replicase polyprotein comprising at least one proteinase cleavage site that exhibits reduced or no cleavage.

35. A nidovirus replicase polyprotein comprising at least one proteinase cleavage site that exhibits reduced or no cleavage.

36. A vaccine comprising (a) a live, attenuated virus of the order Nidovirales, said virus characterized as comprising a genome encoding a replicase polyprotein comprising at least one proteinase cleavage site that exhibits reduced or no cleavage, and (b) a pharmaceutically acceptable diluent.

37. The vaccine of claim 35, wherein said vaccine is formulated as a unit dose of 10⁶ to 10¹⁴ infectious particles.

38. The vaccine of claim 35, wherein said unit dose is provided in a 100 ml aliquot.

39. The vaccine of claim 35, further comprising a preservative.

40. The vaccine of claim 35, wherein said vaccine is lyophilized.
